(19) United States
(12) Patent Application Publication
(10) Pub. No.: US 2023/0225960 A1
(43) Pub. Date: Jul. 20, 2023

(54) Optimized Topical Formulation for Lidocaine Pain Relief Gel

(71) Applicant: Focus Consumer Healthcare, LLC, Chattanooga, TN (US)

(72) Inventor: Jason Bernard Sondgerroth, Chattanooga, TN (US)

(73) Assignee: Focus Consumer Healthcare, LLC, Chattanooga, TN (US)

(21) Appl. No.: 18/098,014

(22) Filed: Jan. 17, 2023

Related U.S. Application Data
(60) Provisional application No. 63/300,265, filed on Jan. 18, 2022.

(51) Int. Cl. A61K 9/00; A61K 31/167; A61K 47/18; A61K 47/36; A61K 47/38; A61K 36/534; A61K 31/198; A61K 31/045; A61K 9/06

(52) U.S. Cl. CPC A61K 9/0014; A61K 31/167; A61K 47/186; A61K 47/36; A61K 47/38; A61K 36/534; A61K 31/198; A61K 31/045; A61K 9/06

(57) ABSTRACT

A moisturizing composition for delivery of lidocaine is provided with viscosity thickening provided by the use of sclerotium gum and hydroxyethylcellulose in approximately equivalent amounts in a slightly acidic vehicle without anionic compounds.

### CLAIM OF PRIORITY

The present application claims the benefit of U.S. Provisional Patent Application No. 63/300,265 filed Jan. 18, 2022.

### FIELD OF THE INVENTION

The invention relates to an improved vehicle for the topical application of lidocaine especially for application to the lips.

### BACKGROUND OF THE INVENTION

Lidocaine is a well-known ingredient that is used topically to provide a local anesthetic affect that may stop itching and pain from certain skin conditions such as scrapes, minor burns, eczema, insect bites and cold sores. Lidocaine, applied topically at concentrations that range from about 1% to 5% has been well tolerated for decades with only rare occurrences of serious allergic reactions or side effects.

The skin that forms human lips is typically thinner and more sensitive than skin on a person's arms and legs because it is used to assist the mouth intake and absorb food. The lips have many nerve endings and do not contain oil glands, unlike skin on other parts of the body. The result is that lips are not only very sensitive, but also predisposed to dryness and chapping. Lidocaine is particularly useful anesthetic for application to the lips since it has less drying affect than menthol and does not create a burning sensation as may result with various capsaicin products.

The problem addressed is the providing for delivery of lidocaine and desired supplementary ingredients in an optimal vehicle for delivery to the lips. It can be readily understood that an optimal vehicle must satisfy many criteria. Important among these are: stability, as products should have an effective shelf life of two years or longer; providing appropriate level of penetration or absorption of the active ingredient and at the desired location; providing appropriate moisturizing and protective benefits to the skin; and providing sensory acceptance of a desirable feel or scent. In addition, the delivery vehicle should accomodate consumer preferences for particular ingredients or classes of ingredients such as vitamins, emollients, organic ingredients, as well as transparency with respect to social, health, environmental, safety issues.

One aspect of the present invention is the use of scleroglucan, commonly referred to as sclerotium gum, as a gelling agent. Scleroglucan is a microbial polysaccharide and this allows for manufacture in bioreactors without the disadvantages of using petroleum-based polymers or polymers of plant origin. Scleroglucan is a high molecular weight non-iconic branched glucan. The non-iconic branched glucan consists in a backbone of (1,3)-β-linked D-glucopyranosyl residues bearing a single (1,6)-β-linked D-glucopyranosyl unit every three sugar residues of the main chain. The length of the polymer chain, and the molecular mass of scleroglucan may differ depending on the microbial strain, fermentation process and the recovery method used in manufacture. Sclerotium gum has been recognized in skin care and cosmetic field as serving as a highly effective suspension agent useful in formulations containing pigments such as zinc or titanium, or other formulations with particles that should be held in suspension and without creating sedimentation issues. In many cases sclerotium gum is used as a thickening agent or as an adjunct with the xanthan gum rather than as a primary gelling agent.

Another aspect of the present invention is the use of benzethonium chloride as an anti-microbial agent. More commonly in topical formulations anti-microbials may be alcohol, parabens, triclosan, and other anionic or neutral agents. Benzethonium chloride is preferred for the present topical application for use on the lips because of its inherently low likelihood of causing skin irritation. This is particularly important on the lips, and especially on lips that may already be damaged and therefore predisposed to further irritation.

The combination of lidocaine and sclerotium gum appears to provide fortuitous benefits. Lidocaine is naturally fast acting but has a relatively short half-life of efficacy. For this reason, among others, lidocaine is often administered in liposome delivery vehicles. Liposomes which delay the release of some portion of the lidocaine and thereby prolong the efficacy of the lidocaine relief are relatively costly. Liposome composition must also be optimized for delayed release instead of sensory appeal. In addition, the liposome material consumes a substantial volume of the delivery formulation that could be optimally used for other active and sensory ingredients. In other typical lidocaine formulations, carbomer (acrylates/C10-30 alkyl acrylate cross polymer) is used as the primary thickening or gelling agent. Since it is desirable to provide non-encapsulated lidocaine in a thickened or gelled vehicle, and carbomers will gel or thicken best in formulations across a broad pH range, these high molecular weight polymers of acrylic acid crosslinked with allyl ethers of polyalcohols have been broadly used in topical cosmetic and excipient delivery vehicles. Carbomer is also the relatively rare petro-chemical based ingredient with minimal safety concerns or warnings. However, carbomer introduces a neutralizing agent into the product formulation since the preparation of carbomer thickened or gelled vehicles involves both a dispersion phase and a subsequent neutralizing phase using neutralizing agents such as triethanolamine (TEA), sodium hydroxide and potassium hydroxide. While such neutralizing agents are considered safe in the concentrations used some users will have both social and health aversions to these ingredients.

### DETAILED DESCRIPTION OF THE INVENTION

The present invention is directed to the formulation and creation of a lidocaine mixture specifically adapted for application to the lips in a gel or lotion form. According to the invention, a formulation is substantially water-based and includes sclerotium gum and hydroxyethylcellulose as primary gelling or thickening ingredients and benzethonium chloride as an antimicrobial. In addition, the formulation preferably includes moisturizers flavorings or sensory ingredients, and ancillary actives to provide anti-inflammatory effects as well as alleviation of cold sore symptoms.

In a preferred formulation, water may comprise about 50-80% of the product by weight. Moisturizers may include hydrolyzed jojoba esters, ethylhexylglycerin, glycerin, and allantoin. Moisturizers will preferably comprise about 15-20% by weight of the formulation.

Lysine HCL and melissa officinalis extract (balm mint) may be provided to help address cold sore symptoms at concentrations of about 4-6%. An anti-inflammatory such as those provided under the Symrise brand, with bisabolol and zingiber officinale (ginger) root extract provide anti-inflammatory treatment at concentrations of under 1%. Flavorings such as peppermint oil and spearmint oil have combined concentrations of less than 1% also improve sensory experience and provide some antimicrobial effect. Benzethonium chloride is provided at concentrations of generally less than 1% (0.01 to 0.2%) for antimicrobial action. The combination of hydroxyethylcellulose and sclerotium gum, in generally equivalent amounts and aggregate concentrations of between about 2% and 5% provide gelling and thickening. The formulation remains substantially free of anionic compounds.

The formulation attempts to provide an optimal viscosity in the range of about 40,000 to 70,000 CPS and tolerable viscosity range of about 30,000 to 100,000 CPS, with the most preferred viscosity range being between about 45,000 to 60,000 CPS. The higher the viscosity, the greater the resistance to flow and measurements of centipoise (CPS) translates so that the 40,000 to 70,000 CPS optimal range is roughly equivalent to the thickness of ketchup or mustard, a thick flowable syrup might have CPS between 5,000 and 25,000 while peanut butter or tomato paste might have a CPS in the range of about 200,000. The viscosity ranges of the preferred formulation are significantly higher than topical skin lotion which might have a viscosity of only 1000 to 2000 CPS. Centipoise is generally measured according to ISO2555 to determine the “Brookfield” viscocity.

Leading lidocaine topical lotion, cream, and gell products in the consumer healthcare market are thickened with carbomer and neutralizing agents. For instance:

| Product Name | Carbomer | Neutralizing Agent |
|---|---|---|
| Lidocane Plus 4% | Yes | triethanolamine |
| Max Strength | acrylates/c10-30 | phosphoric acid |
| Aspercreme with 4% | alkyl acrylate | [ceteth-20 and |
| | crosspolymer | dicetyl phosphate] |
| Blue Emu Lidocaine | Acylates acrylamide | triethanolamine |
| Cream | copolymer | |
| Uber Numb 5% | Carbomer | triethanolamine |
| Gold Bond w/4% | acrylates/c10-30 | phosphoric acid |
| Lidocaine | alkyl acrylate | [ceteth-20 and |
| | crosspolymer | dicetyl phosphate] |
| Icy Hot Lidocaine | acrylates/c10-30 | phosphoric acid |
| plus Menthol | alkyl acrylate | [ceteth-20 and |
| | crosspolymer | dicetyl phosphate] |

The initial attempts to formulate a suitable lidocaine vehicle utilized Carbopol branded carbomer at concentrations of up to 12%, and including additional agents such as hydroxyethylcellulose and polyurethane-based thickeners (Rheoluxe 812) so for instance formulation with 8% Carbopol Aqua CC (polyacrylate-1 crosspolymer) and 0.5% hydroxyethylcellulose did not thicken. A 12% Carbopol Aqua CC formulation did not thicken and was not clear. A 12% Carbopol Aqua CC with an additional 2% Rheoluxe 812 did not thicken and was not clear. These results, suggested by the prior art lidocaine formulations were obviously unsatisfactory, even with these very substantial levels of thickening agents.

Initial attempts to utilize either hydroxyethylcellulose or sclerotium gum were also unsatisfactory. Hydroxyethylcellulose alone resulted in a stringy composition rather than a thick and smooth lotion while the use of sclerotium gum alone created a more jelly-like vehicle. Neither was deemed suitable for a topical vehicle for application to the lips. Further attempts to utilize both hydroxyethylcellulose and sclerotium gum with concentrations of at least 0.5% of each provided some thickness and were clear.

However, it was not until the concentrations of hydroxyethylcellulose and sclerotium gum were provided in roughly equivalent amounts, each in excess of 1% by weight, that a satisfactory level of thickening became apparent. The concentrations of hydroxyethylcellulose and sclerotium gum will also generally each be less than 4% by weight. The weight of sclerotium gum should be within about 25% (plus or minus) of the weight of hydroxyethylcellulose, and preferably within about 15%.

In order to prepare a formula according to the invention it is desirable to proceed through a multi-step process with at least four separate groups of ingredients. A first order-based group of ingredients will include water, possibly aloe barbadensis leaf juice, disodium EDTA and the sclerotium gum. This first group of ingredients is mixed in a main vessel at RPMs greater than 300 revolutions per minute and heated to approximately 60-65 degrees Centigrade. When this temperature is obtained, the first group is mixed with high shear (in excess of 400 RPM) for approximately 15 minutes or until the sclerotium gum is hydrated and then the mixture can be cooled to 40-45 degrees Centigrade. In the main vessel, the second group of ingedients is added including hydrolyzed jojoba esters and ethylhexyglycerine moisturizers , along with lysine HCL and lidocaine HCL followed by the essential oils for flavoring and anti-inflammatories. This is mixed at 40-45 degrees and the pH is adjusted to about 6. pH is required to be between 5.5 and 6.5. When the pH is appropriately adjusted, then the third group of benzethonium chloride is added to the mixture.

In an auxiliary vessel, fourth group of ingredients including glycerin, is mixed with allantoin and fully dispersed before hydroxyethylcellulose is added. This fourth group is not utilized until the final step. Then, the auxiliary vessel is added to the main vessel containing the first three groups of ingredients, and mixed for over thirty minutes at 40 to 45 degrees Centigrade. Subsequently, the final mixture can be packaged in squeeze tubes or other appropriate containers for distribution and use.

Numerous alterations of the structure herein disclosed will suggest themselves to those skilled in the art. However, it is to be understood that the present disclosure relates to the preferred embodiment of the invention which is for purposes of illustration only and not to be construed as a limitation of the invention. All such modifications which do not depart from the spirit of the invention are intended to be included within the scope of the appended claims.

What is claimed herein is:

1. A topical composition comprising: from about 0.5% to about 4.0% by weight of lidocaine or lidocaine hydrochloride; from about 0.1% to 0.5% by weight of benzethonium chloride; from about 0.3% to about 3% by weight of a polysaccharide sclerotium gum thickening agent; and from about 30% to about 90% by weight of an aqueous carrier.

2. The moisturizing composition of claim 1 further comprising from about .03% to about 3% by weight of hydroxyethylcellulose.

3. The topical composition of claim 2 wherein the ratio of sclerotium gum thickening agent to the hydroxyethylcellulose is between about 0.85 to 1.15.

4. The composition of claim 1, wherein the composition has a form selected from the group consisting of emulsions, gels, and combinations thereof.

5. The composition of claim 2, further comprising from about 0.01% to about 20.0% by weight of anti-inflammatory agents, flavorings, moisturizers, and combinations thereof.

6. The composition of claim 2, wherein said composition has a viscosity of from about 30,000 to about 100,000 centipoise.

7. The composition of claim 1, wherein said composition is substantially free of anionic compounds.

8. The composition of claim 2, wherein said composition has a viscosity of from about 40,000 to about 70,000 centipoise.

9. The composition of claim 2, further comprising up to about 6% cold sore mitigating ingredients.

10. The composition of claim 9 wherein the cold sore mitigating ingredients are selected from Lysine and balm mint.

11. A method of manufacturing a topical composition comprising the steps of preparing and mixing a first group between 30 to about 90% by weight of water with between 1% to about 3% by weight of sclerotium gum until the sclerotium gum is hydrated;
adding a second group of ingredients including moisturizers and lidocaine to the first group and adjusting the pH of the combined ingredients to between 5.5 and 6.5;
adding benzethonium chloride in an amount up to 0.2% by weight to the combined groups of ingredients;
preparing a third group of ingredients including glycerin and hydroxyethylcellulose;
adding the third group of ingredients to the sclerotium gum and moisturizing groups and mixing.

12. The method of claim 11 wherein the ratio of the weight of sclerotium gum to the weight of the hydroxyethylcellulose is between 1:0.75 and 1:1.25.

13. The method of claim 11 wherein the second group of ingredients includes up to about 6% cold sore mitigating ingredients.

14. The method of claim 13 wherein the cold sore mitigating ingredients include lysine.

15. The method of claim 12 wherein the resulting mixture has a viscosity of from about 30,000 to about 100,000 centipoise.

16. The method of claim 12 wherein the resulting mixture is a gel or lotion having a viscosity of from about 45,000 to about 60,000 centipoise.

17. The method of claim 12 wherein the second group of ingredients includes up to 1% of anti-inflammatory actives.

18. The method of claim 17 wherein the anti-inflammatory actives include bisabolol.

19. The method of claim 12 wherein the second group of ingredients includes less than 1% of an essential oil selected for scent or flavor.

20. The method of claim 16 wherein the gel or lotion is packaged in squeeze tubes for distribution and use.
